(19) United States
(12) Patent Application Publication
(10) Pub. No.: US 2011/0040331 A1
(43) Pub. Date: Feb. 17, 2011

(54) POSTERIOR STABILIZER

(76) Inventors: Jose FERNANDEZ, Coral Springs, FL (US); Kevin REDMOND, Lighthouse Point, FL (US)

Correspondence Address: CHRISTOPHER & WEISBERG, P.A., 200 EAST LAS OLAS BOULEVARD, SUITE 2040, FORT LAUDERDALE, FL, 33301, US

(21) Appl. No.: 12/784,065

(22) Filed: May 20, 2010

Related U.S. Application Data
(60) Provisional application No. 61/179,873, filed on May 20, 2009.

(51) Int. Cl. A61B 17/70

(52) U.S. Cl. 606/264; 606/246

(57) ABSTRACT

A dynamic hybrid stabilization system having a plurality of affixation elements coupled to a spinal segment having at least two intervertebral spaces, each affixation element having a medially extending connector element. A plurality of dynamic stabilization devices having an adjustable range of motion and being spaced medially from the affixation elements is included, each dynamic stabilization device being coupled to at least one of the connector elements and including an attachment element spanning at least one intervertebral space, such that each dynamic stabilization device and the connector element when coupled together span at least two intervertebral spaces.

### CROSS-REFERENCE TO RELATED APPLICATION

This application is related to and claims priority to U.S. Provisional Patent Application Ser. No. 61/179,873, filed May 5, 2009, entitled POSTERIOR STABILIZER, the entirety of which is incorporated herein by reference.

### STATEMENT REGARDING FEDERALLY SPONSORED RESEARCH OR DEVELOPMENT

n/a

### FIELD OF THE INVENTION

The present invention relates to spinal prostheses, and in particular, a method and system for spinal stabilization, including prostheses and components implantable on or about a spinal segment.

### BACKGROUND OF THE INVENTION

A significant portion of the population experiences pain or discomfort resulting from spinal injuries or degenerative conditions in and around the vertebral discs. While many individuals may simply experience minor sprains or strains that may be somewhat limiting, numerous individuals may develop severe lower back pain caused by inflammatory changes in the lumbar disc associated with such changes.

A spinal segment includes one or more vertebrae, intervertebral discs and facet joints providing flexion and extension movement of spine. Degenerative changes in the intervertebral discs can lead to changes in the facet joint, and vice versa. In order to treat a degenerative condition and to alleviate the pain involved with such a malady, surgical methods may be employed to replace the degenerative component of the spinal segment, such as the damaged disc. However, the replacement of a degenerative disc may not suffice, as the facet joint components of the spinal column may still be a source of discomfort and/or limited mobility. As such, it may desirable to replace a degenerative or problematic facet joint with a posterior stabilization device.

In addition, the degenerative process may result in a condition called spinal stenosis, where there is a narrowing of the spinal canal. This is caused by a combination of reduced disc height, ligamnetum hypertrophy, a forward slip of the vertebra and disc bulging. Surgery is sometime needed to deal with this condition, where a surgical procedure may involve decompression of the spine and/or the removal of posterior portions of the spinal column. This often makes the spine unstable, requiring stabilization of the spine after the decompression. Such stabilization may be achieved with an instrumented postero-lateral fusion, where pedicle screws are inserted into the vertebra to be fused and connected with rods or plates, and bone is laid on the side of the spine over the transverse processes. Stabilization may also be accomplished by a dynamic stabilization device where there is no need to add a fusion, and the device stabilizes the spine. A posterior dynamic stabilization device is typically attached to pedicle screws inserted in to the vertebrae, however, conventional devices are limited in their allowable range of motion and results may vary from patient to patient. Where multiple discs have degenerated, it may further be desirable to stabilize multiple, consecutive segments of the spine in an effort to treat an entire affected region with a combination of fixation devices such as rods as well as providing desirable ranges of motions with one or more dynamic stabilizers in adjacent areas of the spinal segment.

Accordingly, it would be desirable to provide a hybrid spinal stabilization system having fixed and movable components that can stabilize a segment of the spinal column spanning multiple intervertebral discs by limiting the range of motion in one segment of a spinal column while providing increased ranges of movement in adjacent segments of the spinal column.

### SUMMARY OF THE INVENTION

The present invention advantageously provides a method and system for spinal stabilization. In particular, the spinal stabilization system may include both fixed and movable components providing a minimum reduction in the natural movement of a first motion segment of the spinal column while preventing or restricting movement in a second motion segment of the spinal column when implanted.

In particular, a spinal stabilization system is provided, including a posterior stabilization device including a first segment engageable with a first and second vertebra, the first segment providing a range of motion allowing at least one of the first and second vertebra to articulate with respect to the other, and a second segment engageable with the second vertebra and a third vertebra, the second segment substantially preventing movement of the second and third vertebrae with respect to one another. The system may also include a plurality of affixation elements engageable with the posterior stabilization device to secure the posterior stabilization device to the first, second, and third vertebrae, where at least one of the affixation elements defines a head movably coupled to a threaded shaft. The system may include a connector element, the connector element defining a collar engageable with the posterior stabilization device and an elongate portion engageable with an affixation element, where the connector element defines a joint providing movement between the collar and the stabilization device. An articulating intervertebral prosthesis may be positionable between the first and second vertebrae, and a non-articulating intervertebral prosthesis may be positionable between the second and third vertebrae. The first segment of the posterior stabilization device may define an arcuate range of motion, and the range of motion of the first segment of the posterior stabilization device may be selectively adjustable. The first segment of the posterior stabilization device may define a joint having at least two degrees-of-freedom of movement, and may include at least three degrees-of-freedom of movement. The first segment of the posterior stabilization device may include at least one resistive element providing resistance for at least a portion of the range of motion of the first segment. The second segment of the posterior stabilization device may include an elongate rod extending from the first segment. In addition, the first segment of the posterior stabilization device may include a first component movably coupled to a second component, and the second segment of the stabilization device may include an elongated portion of the second component.

A spinal stabilization system is also provided, including a first posterior stabilization device including a first component engageable with a first vertebra and a second component engageable with a second vertebra, where the first and second components are movably coupled to one another to define a range of motion between the first and second vertebra, and where the second component is engageable with a third vertebra to substantially prevent movement between the second and third vertebrae; a first affixation element anchoring the first component to the first vertebra; a second affixation element anchoring the second component to the second vertebra; and a third affixation element anchoring the second component to the third vertebra. The system may include a connector element defining a collar movably coupled to the first component and an elongate portion coupled to the first affixation element. The first and second components of the stabilization device may be movably coupled to one another to define a joint having at least three degrees-of-freedom.

A spinal stabilization system is also disclosed, including a first posterior stabilization device including a first component engageable with a first vertebra and a second component engageable with a second vertebra, where the first and second components are movably coupled to one another to define a range of motion between the first and second vertebra, and where the second component is engageable with a third vertebra to substantially prevent movement between the second and third vertebrae; a first affixation element anchoring the first component to the first vertebra; a second affixation element anchoring the second component to the second vertebra; a third affixation element anchoring the second component to the third vertebra; a second posterior stabilization device including a third component engageable with the first vertebra and a fourth component engageable with the second vertebra, where the third and fourth components are movably coupled to one another to define a range of motion between the first and second vertebra, and where the fourth component is engageable with the third vertebra to substantially prevent movement between the second and third vertebrae; a fourth affixation element anchoring the third component to the first vertebra; a fifth affixation element anchoring the fourth component to the second vertebra; a sixth affixation element anchoring the fourth component to the third vertebra.

### BRIEF DESCRIPTION OF THE DRAWINGS

A more complete understanding of the present invention, and the attendant advantages and features thereof, will be more readily understood by reference to the following detailed description when considered in conjunction with the accompanying drawings wherein:

FIG. 1 is a first view of an embodiment of a dynamic connector constructed in accordance with the principles of the present invention;

FIG. 2 is a second view of the dynamic connector shown in FIG. 1;

FIG. 3 is a cross-sectional view of the dynamic connector shown in FIG. 1;

FIG. 4 is a third view of the dynamic connector shown in FIG. 1;

FIG. 5 is a perspective view of an embodiment of a posterior dynamic stabilization device;

FIG. 6 is an assembly view of the posterior dynamic stabilization device shown in FIG. 5;

FIG. 7 is a cross-sectional view of the posterior dynamic stabilization device shown in FIG. 5;

FIG. 8 is a perspective view of another embodiment of a posterior dynamic stabilization device constructed in accordance with the principles of the present invention;

FIG. 9 is an illustrative view of the posterior dynamic stabilization device shown in FIG. 8;

FIG. 10 is a side view of the posterior dynamic stabilization device shown in FIG. 8;

FIG. 11 is a perspective view of another embodiment of a dynamic connector constructed in accordance with the principles of the present invention;

FIG. 12 is a cross-sectional view of the dynamic connector shown in FIG. 11;

FIG. 13 is a perspective view of still another embodiment of a dynamic connector constructed in accordance with the principles of the present invention;

FIG. 14 is an additional perspective view of the dynamic connector shown in FIG. 13;

FIG. 15 is a perspective view of a hybrid spinal stabilization system constructed in accordance with the principles of the present invention;

FIG. 16 is an additional perspective view of a hybrid spinal stabilization system constructed in accordance with the principles of the present invention;

FIG. 17 is still another perspective view of a hybrid spinal stabilization system constructed in accordance with the principles of the present invention;

FIG. 18 is a perspective view of another embodiment of a dynamic posterior stabilization device constructed in accordance with the principles of the present invention;

FIG. 19 is a cross-sectional view of the dynamic posterior stabilization device shown in FIG. 18.

FIG. 20 is another cross-sectional view of the dynamic posterior stabilization device shown in FIG. 18.

FIG. 21 is an exploded view of a portion of the dynamic posterior stabilization device shown in FIG. 20;

FIG. 22 is a posterior view of the extension and contraction of the dynamic posterior stabilization device shown in FIG. 18;

FIG. 23 is a lateral view of the extension and contraction of the dynamic posterior stabilization device shown in FIG. 18; and

FIG. 24 is a posterior view of the angular range of motion of the dynamic posterior stabilization device shown in FIG. 18.

### DETAILED DESCRIPTION OF THE INVENTION

The present invention includes a spinal stabilization system implantable on or about one or more spinal segments spanning one or more intervertebral discs and vertebrae. In particular, the spinal stabilization system may include several stabilizing components having varying ranges of movement and/or degrees-of-freedom to selectively provide varying ranges of motion for adjacent portions of a spinal segment. By combining one or more of the following described components about a particular vertebral site (which may include one or more intervertebral discs, vertebrae, and facet joints), a desired range of spinal motion and/or level of fixation may be selected and achieved.

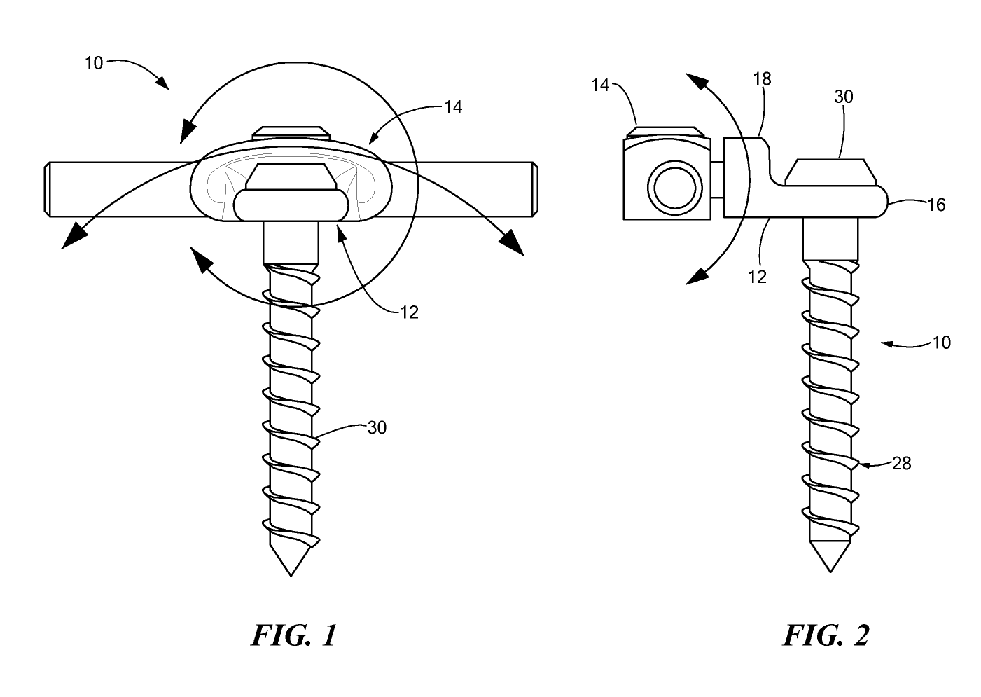

Now referring to the figures, in which like reference designators refer to like elements, there is shown in FIGS. 1-4 an embodiment of a dynamic connector constructed in accordance with the principles of the present invention and generally designated as “10.” The illustrated dynamic connector 10 may generally provide a desired degree or range of motion between a fusion, fixation rod, or other spinal prosthesis and a pedicle screw or other anchoring component, as discussed in more detail below.

The dynamic connector 10 may generally include a first connective component 12 that movably attaches or couples to a second connective component 14. Particularly, the first connective component 12 may generally define an L-shaped configuration (as shown in the side view of FIG. 2), which includes a flat portion 16 for receiving an anchor, such as a pedicle screw, and a substantially cylindrical or disc-shaped portion 18.

The connection between the first connective component 12 and the second connective component 14 may generally provide a desired degree of movement, including selective rotative, translational, and separation capabilities. These movable characteristics may be achieved by a joint 20 defined or otherwise included between the two respective components. For example, the first connective component 12 may include a socket 22 in the disc-shaped portion 18 that couples to a head 24 of the second connective component 14. The head 24 may be rotatably, pivotally, and translationally movable within the socket 22 of the first connective component 14 as indicated by the directional arrows in FIGS. 1-3. The socket 22 may be substantially curved in shape to accommodate a variety of differently shaped and sized heads 24 resulting in a desired range of movement, whether rotational and/or translational. For example, the first connective component 12 may pitch, yaw, or roll about the second connective component 14 in addition to allowing some translational movement (e.g., moving towards and away from the second component 14), thus providing at least four degrees of freedom of movement.

The range of motion between the first connective component 12 and the second connective component 14 may be selectively limited, by example, through the implementation of one or more bumpers 26 disposed at least partially within the socket 22 of the first connective component 12. The bumpers 26 may be constructed from a flexible or dampening polymer to absorb forces experienced upon implantation and/or to limit or restrict the range of motion between the components of the device 10. The bumpers 26 may further be sized and shaped to provide for a particular range of motion. For example, the bumpers 26 may be substantially spherical or ovoid in shape depending on the desired range of movement. The particular range and/or degree of motion may also be manipulated or otherwise affected by the particular shape or dimensions of the socket 22, head 24, and or other portions of the respective first connective component 12 and second connective component 14.

As indicated above, the first connective component 12 may receive an affixation element 28, such as a pedicle screw, or the like, that affixes or otherwise attaches at least a portion of the dynamic connector 10 to part of a spinal segment, such as a pedicle, vertebrae, or the like. For example, to facilitate attachment or fixation between the affixation element 28 and the first connective component 12, the first connective component 12 may define an aperture or opening (not shown) in a face or portion thereof, allowing passage of a portion of the affixation element 28 there through for subsequent anchoring into the spinal segment. The affixation element 28 may further define a head 30 having a larger diameter than that of the opening while a threaded portion of the anchoring element passes through the opening. The head 30 of the affixation element 28 may define a shaped recess 32 for insertion of a screw driver, or similar tool.

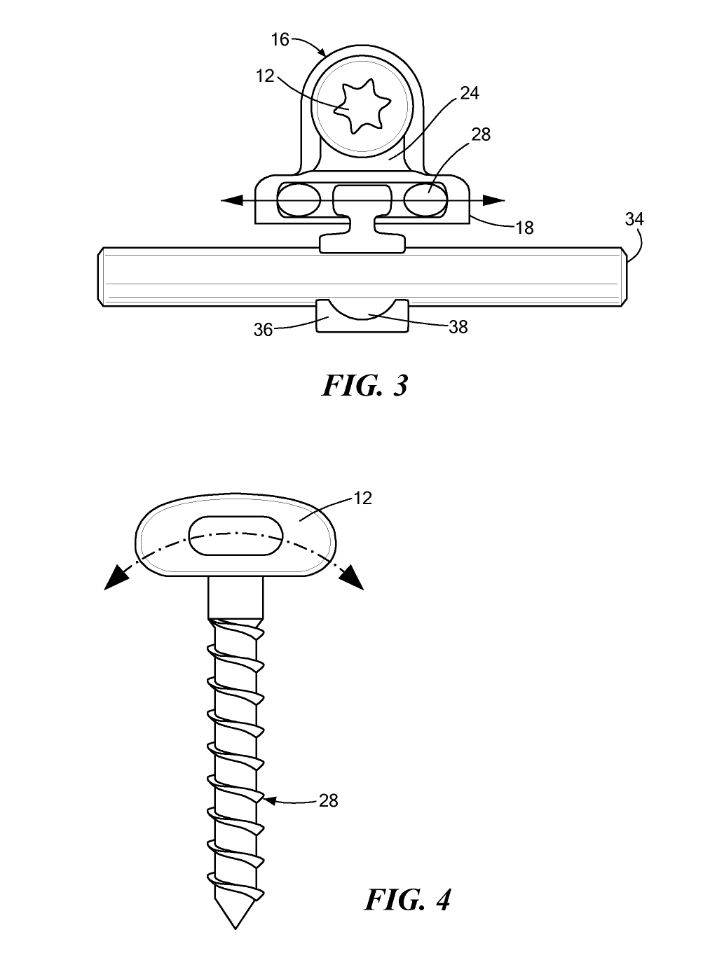

The second connective component 14 may further be attachable or otherwise coupled to an implantable component or prosthesis 34, such as spinal rod. The second connective component 14 of the stabilizing device 10 may define a passage 36 there through for receiving the rod 34. The passage 36 may include a tightening mechanism (such as a set screw or the like, not shown) to secure the engagement and position of the rod 34 relative to the second connective component 14. A compression or friction fit may be equally applicable to secure the assembly. In addition, there may be a selectively adjustable tightening mechanism to allow for some degree of movement or translation between the second connective component 14 and the rod 34 upon implantation. For example, as shown in FIG. 3, the rod 34 is slideably received within the passage 36, which is substantially curved in shape to accommodate a substantially cylindrical rod 34. The second connective component 14 may further define flared portions 38 that extend over the passage 36 to aid in securing the rod 34 to the second connective component 14. The flared portions 38 may define a substantially curved inner surface to facilitate the translational movement of the rod 34 with respect to the second connective component 14 while preventing the detachment or disengagement of the rod 34 to the passage/second connective component 14.

One or more dynamic connectors 10 may be implanted or otherwise inserted into one or more spinal segments to provide the desired range and degree of motion. For example, it is contemplated that a plurality of dynamic connectors 10 may be inserted to a plurality of sequential or consecutive pedicles or vertebrae along a length of troubled or surgically targeted spinal column for repair, where the devices are connected to one another by one or more connecting rods or other implantable prostheses.

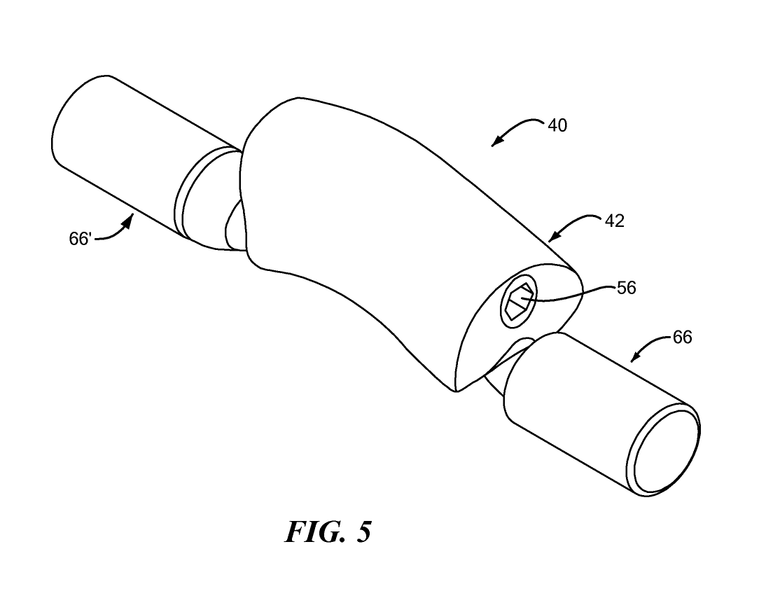

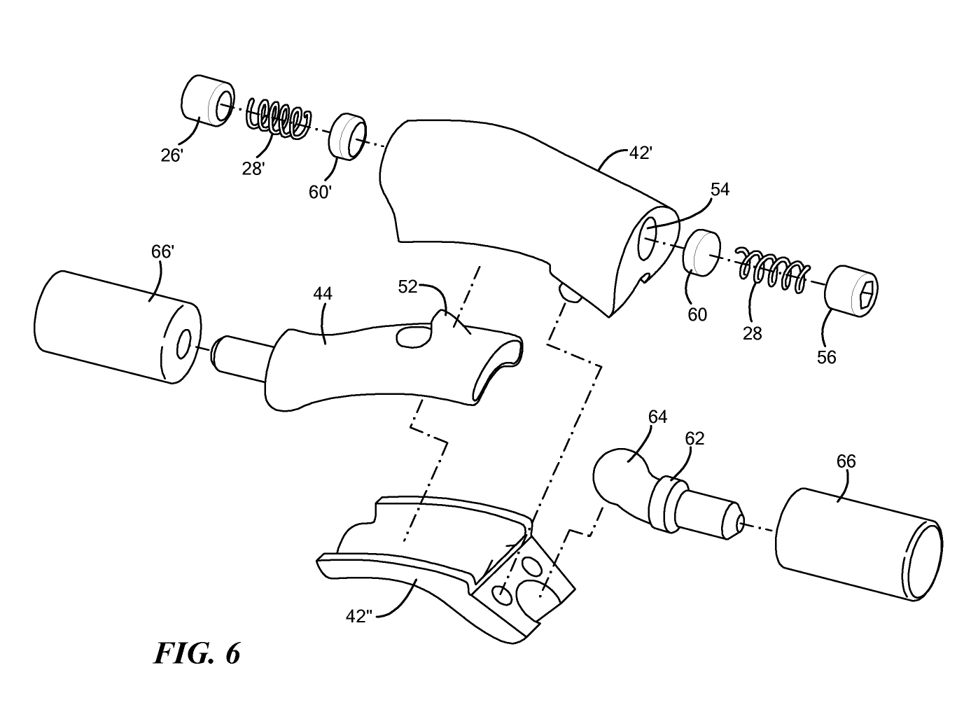

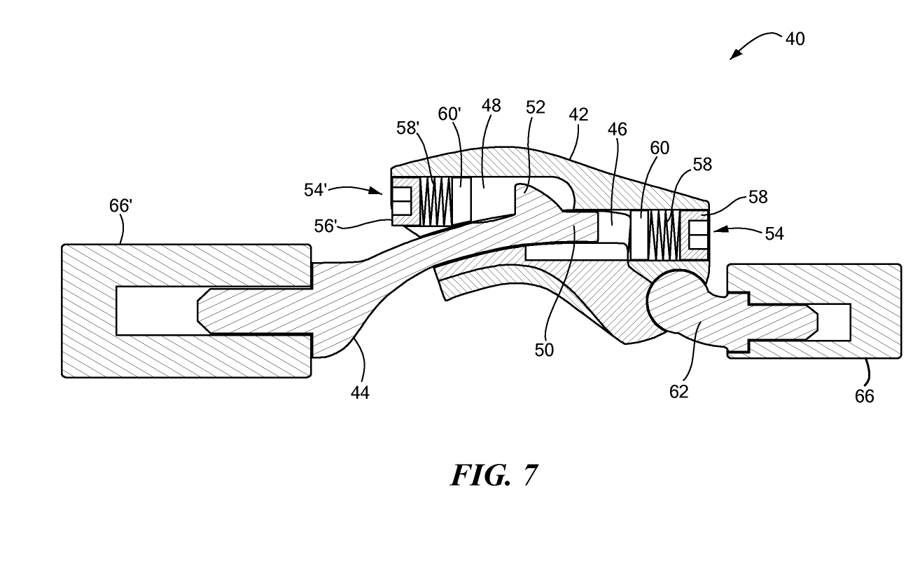

Now referring to FIGS. 5-7, a posterior dynamic stabilization device 40 is shown generally including a first component 42 and a second component 44, where the first and second components are movably coupled to one another to define a range or path of motion there between. As used herein, the term “path of motion” is intended to include a length, distance and/or shape associated with the movement of either and/or both the first and second components 42, 44. The motion of the first component 42 and second component 44 may include an arcuate path about which the first and second components 42, 44 are able to articulate, where the arcuate path may define a point of rotation about which the first and second components 42, 44 move.

In particular, the first component 42 may include a body defining an opening providing access to a first cavity or recessed region 46, where the first cavity 46 is able to receive at least a portion of the second component 44. The body of the first component 42 may further include a second cavity 48 or channel adjacent to or otherwise in proximity to the first cavity 46, where a portion of the first and second cavities may be coupled or otherwise in fluid communication with each other. Further, the first and second cavities may include contoured or arcuate walls extending along at least a portion of their respective lengths. Of note, the first component 42 may be constructed as a unitary element, or alternatively, be composed of multiple parts that are fused, welded, or otherwise assembled together to form the desired characteristics and features of the component. For example, the first component 42 may include a first housing element 42′ and a second housing element 42″ that may be fitted or otherwise coupled together. Moreover, the first component 42 may be constructed from a myriad of biocompatible materials, including metals, plastics, and polymers as is known in the art.

The second component 44 may define a body having an articulating portion 50 positionable within or otherwise movable about the first component 42, where the articulating portion 50 may define an arcuate or contoured shape. For example, the articulating portion 50 of the second component 44 may be movably positionable within the first cavity 46 of the first component 42. The arcuate and/or contoured shapes of both the articulating portion of the second component 44 as well as the walls of the first cavity of the first component 42 may provide an arcuate path or range of motion between the two. In addition, the body of the second component 44 may further include a protrusion 52 extending from the articulating portion, where the protrusion 52 is positionable within the second cavity 48 of the first component 42.

The body of the first and/or second components may further define one or more openings for the insertion or placement of an adjustment element that may be used to manipulate or otherwise modify the path of motion between the first and second components. For example, first and second adjustment openings 54, 54′ may be included on either end of the first component 42 about the first and second cavities providing access thereto. In addition, the dynamic stabilization device 40 may include one or more adjustment elements 56, 56′ positionable within the first and second adjustment openings 54, 54′ to affect the path of motion between the first and second components and/or the behavior and characteristics of the movement. For example, the stabilization device 40 may include one or more set screws that can be adjustably positioned within either and/or both of the first and second adjustment openings 54, 54′ to reduce or enlarge the path of motion between the first and second components. The set screws may be positioned at a desired location within the first component 42 to provide a stop against which the second component 44 comes into contact with during movement to prevent and/or restrict further movement. In addition to the adjustment elements 56, 56′, one or more resistive elements 58, 58′ and/or one or more dampening elements 60, 60′ may be adjustably positionable within either and/or both of the first and second adjustment openings to provide resistance and/or dampening of the forces experienced as the first and second components move relative to one another. The resistive or dampening elements may include springs, washers, a dashpot mechanism, or the like to provide the desired movement characteristics.

The dynamic stabilization device 40 may further provide one or more degrees of freedom of movement to compensate for inaccuracies experienced during implantation and/or to allow the device to adapt to movements of a spinal segment, including the ability to allow flexion/extension, lateral bending, and axial rotation. For example, the dynamic stabilization device 40 may include a joint element 62 movably coupled to one of the first or second components, where the joint element 62 may also include a portion extending from the first and/or second components for attachment to an affixation device or the like. The joint element 62 may include a rounded portion 64 that forms a ball-and-socket joint with either of the first and second components, or may alternatively include a hinge or other movable construct providing one or more degrees-of-freedom of movement.

The dynamic stabilization device 40 may also include one or more attachment elements 66, 66′ for facilitating affixation to the spinal segment. The attachment elements 66, 66′ may include a cylindrical element or extension coupled to either and/or both of the first and second components, where the attachment elements 66, 66′ may be matable with a pedicle screw or other affixation element for implantation of the dynamic stabilization device 40 on a spinal segment.

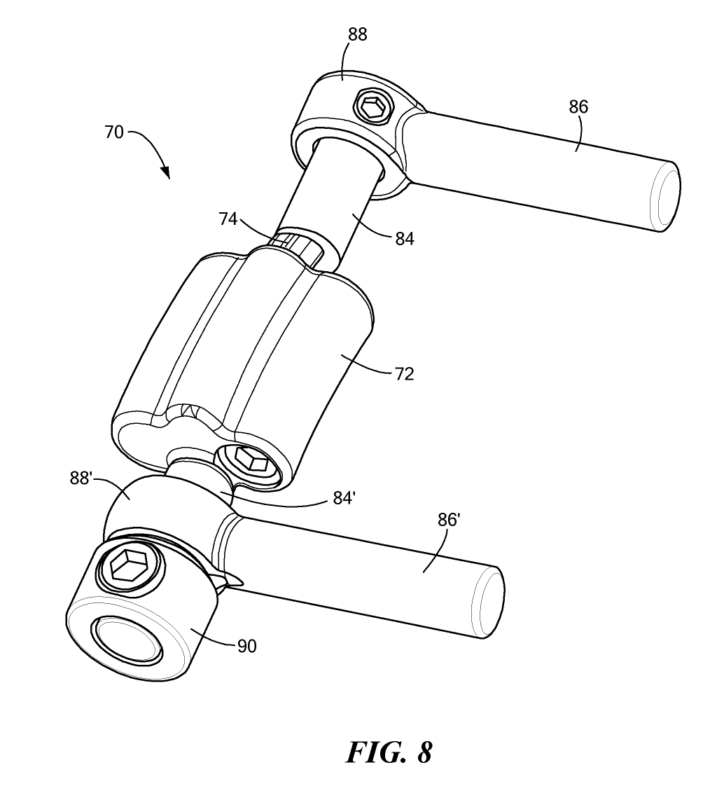

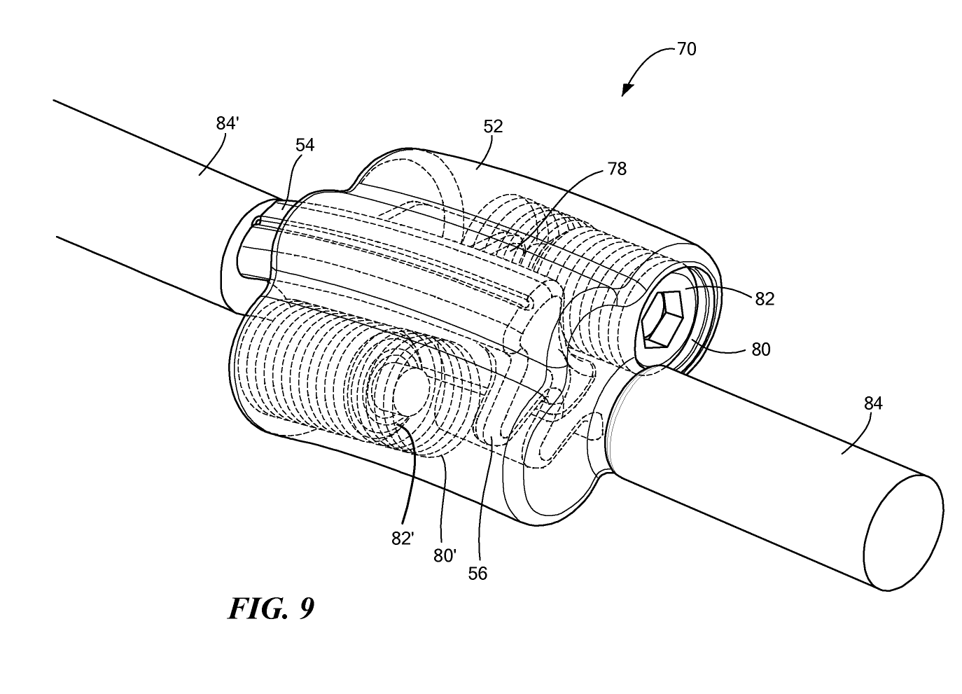

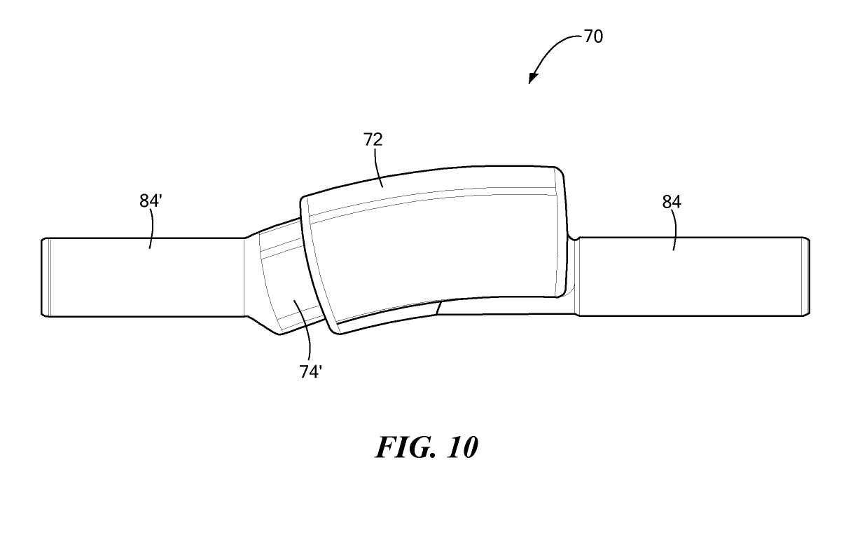

Now referring to FIGS. 8-10, another posterior dynamic stabilization device 70 is shown including a first component 72 and a second component 74 movably coupled to one another to define a path of motion there between, where the path of motion may provide an arcuate path about which the first and second components are able to articulate.

In particular, the first component 72 may include a body defining an opening providing access to a first cavity or recessed region for receiving at least a portion of the second component 74. The second component 74 may define a body having an articulating portion positionable within or otherwise movable about the first component, where the articulating portion may define an arcuate or contoured shape. For example, the articulating portion of the second component 74 may be movably positionable within the first cavity of the first component 72. The arcuate and/or contoured shapes of both the articulating portion of the second component as well as the walls of the first cavity of the first component may provide an arcuate path or range of motion between the two. In addition, the body of the second component may further include a first protrusion 76 extending from the articulating portion, as well as a second protrusion 78 extending from the articulating portion, where both protrusions are positionable within a portion of the first component 72.

The body of the first and/or second components may further define one or more openings for the insertion or placement of an adjustment element that may be used to manipulate or otherwise modify the path of motion between the first and second components. For example, first and second adjustment openings 80, 80′ may be included on either end of the first component providing access to an interior thereof. In addition, the dynamic stabilization device 70 may include one or more adjustment elements 82, 82′ positionable within the first and second adjustment openings to affect the path of motion between the first and second components and/or the behavior and characteristics of the movement. For example, the stabilization device 70 may include one or more set screws that can be adjustably positioned within either and/or both of the first and second adjustment openings to reduce or enlarge the path of motion between the first and second components. The set screws may be positioned at a desired location within the first component to provide a stop against which the first and second protrusions of the second component 74 come into contact with during movement to prevent and/or restrict further movement. In an exemplary embodiment, the path of motion may be adjustable from a length of 0 mm, where motion is restricted, to approximately 8 mm in a particular direction.

The dynamic stabilization device 70 may also include one or more attachment elements for facilitating affixation to the spinal segment. For example, first and second attachment elements 84, 84′ may include cylindrical bodies or extensions coupled to either and/or both of the first and second components, where the attachment elements 84, 84′ may be matable with a pedicle screw or other intermediate elements for implantation of the stabilization device on a spinal segment.

In addition, one or more connector elements may be provided that are movably positionable about the one or more attachment elements 84, 84′ of the stabilization device 70 for affixation to a spinal segment. First and second connector elements 86, 86′ may be included, where the connector elements define collar portions 88, 88′ having an opening there through for movably coupling the connector elements 86, 86′ to the first and second attachment elements 84, 84′, respectively. The collar portions 88, 88′ may include an opening for the insertion of a set screw, which may be adjusted to tighten the collar about the attachment element once the desired positioning has been achieved.

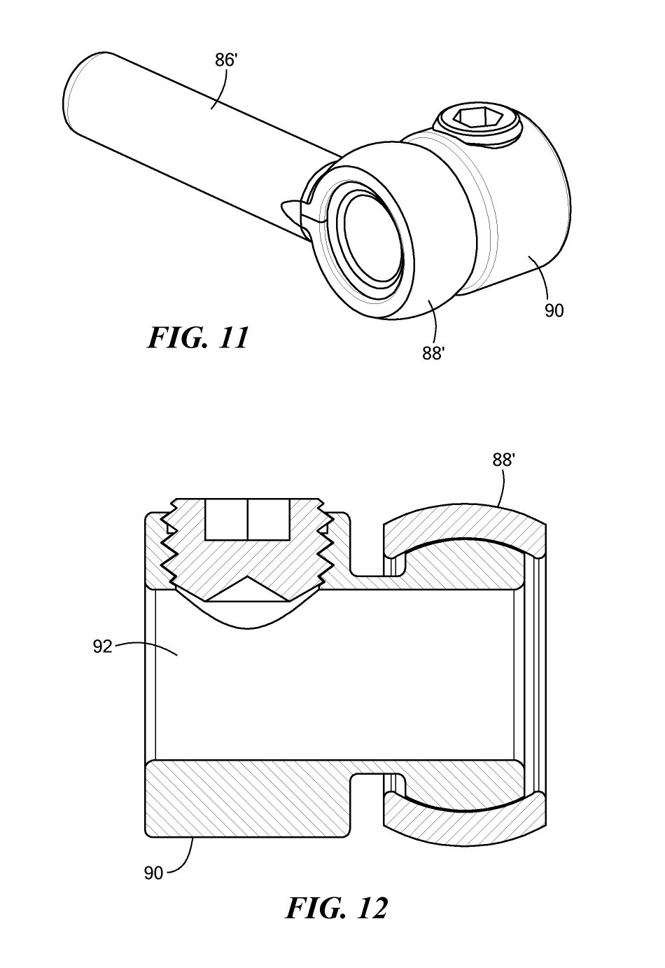

The first and second connector elements 86, 86′ may provide varying ranges of movement with respect to the stabilization device 70. For example, first connector element 86 may be rigidly coupled to the attachment element 84 of the stabilization device 70. As shown in FIGS. 11 and 12, the second connector element 86′ may further include a bearing or joint 90 engageable with the attachment elements 84, 84′ of the stabilization device 70 and the collar portion 88′ to provide for additional movement capabilities between the stabilization device 70 and a spinal segment. In particular, the bearing 90 may define an opening 92 there through for the passage of either the first or second attachment elements 84, 84′ of the dynamic stabilization device 70 (or 40). The bearing 90 may further movably couple to the connector element collar 88′ as to form a ball-and-socket joint, thereby allowing the attachment elements 84, 84′ and thus the first and second components of the stabilization device 40,70 to move and adjust to the forces and motion of a spinal segment, including flexion/extension, lateral bending, and axial rotation.

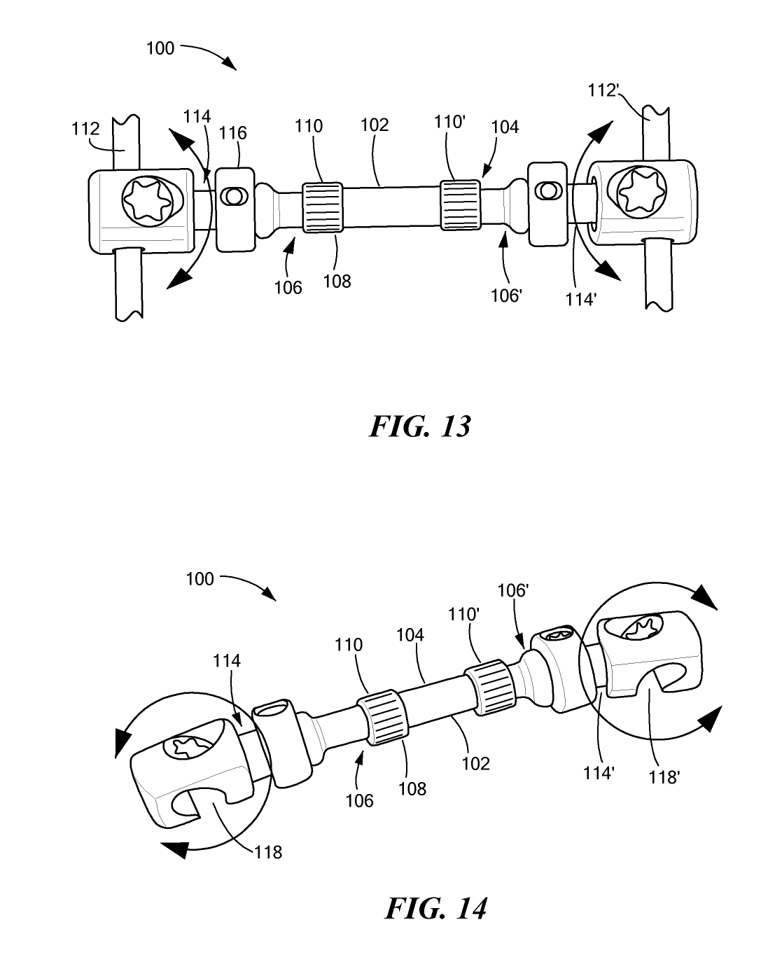

Now referring to FIGS. 13-14, another embodiment of a dynamic connector device for spinal stabilization is shown, generally designated as “100.” The dynamic connector 100 may generally be coupled to two or more implants (such as spinal rods or other prostheses) to provide a desired degree of motion, while also providing a desired amount of fixation or stabilization. The dynamic connector 100 may be comprised of substantially rigid materials, such as stainless steel or titanium, composites, or ceramics. The dynamic connector 100 may generally include a longitudinal or elongated body 102 having an adjustable length that is positionable across a transverse section of a spinal segment. The adjustable length may be provided, for example, by one or more telescoping members 104 selectively adjustable to provide a desired length. For example, as shown in FIGS. 13-14, the telescoping member 104 may include first members 106 and 106′ and a second member 108, each being substantially hollow or solid respectively. The first members 106 and 106′ may be substantially cylindrical in shape and may be slideably positionable within a portion of respective openings of the second member 108, which may also be substantially cylindrical in shape. The first members 106 and 106′, and second member 108, may also be concentric and/or coaxially with respect to each other.

In an exemplary operation, the first members 106 and/or 106′ may be urged or otherwise moved within a portion of second member 108. For example, first members 106 and 106′ may each be slideably received a few millimeters within the second member 108. Alternatively, first member 106 and 106′ may each independently be slideably inserted a desired distance within second member 108, which may be variable depending on the desired width and reach of the dynamic connector 100 to other implants. For example, as shown in FIG. 13, first member 106 is disposed further within the second member 108 than is the first member 106′. The first members 106 and 106′ may either be secured at a desired position within the second member 108 or may be freely movable within the second member 108 in response to movement of the patient.

The first members 106 and 106′ may be optionally releaseably secured within a portion of the second member 108 by fasteners 110 and 110′, which may be independently rotatable about the second member 108. Securing the first members 106 and 106′ at a variety of positions facilitates the dynamic connector 100 having an adjustable length. The adjustable length allows the dynamic connector 100 to connect to two or more rods or prostheses 112, 112′ that may have a varying distance between them from one patient to another. The adjustable length may be securable at any one of the selective lengths to fix the length of the connector 100 once implanted.

The dynamic connector 100 may further include one or more joints 114 that allows a desired degree or range of movement between the dynamic connector 100 and the rods/prostheses 112, 112′. For example, the dynamic connector 100 may include first and second joints 114, 114′ positioned at respective end portions of the dynamic connector 100. The joints 114 and 114′ may include ball-and-socket components, translational and/or pivotal hinge-like constructions, or may contain other adjustable and/or selectively movable mechanisms. Each joint 114 and 114′ may also include a locking or tightening mechanism 116 such as a set screw, movable pin, or the like that can restrict the particular range or degree of motion of either joint 114 and 114′ of the dynamic connector 100. With a selectable length and range of motion at either end of the dynamic connector 100 interfacing with or coupling to the spinal rods or prostheses 112, 112′, the dynamic connector 100 provides stability to a posterior implant construct while also achieving or preserving some natural, physiological motion of the affected spinal segment.

Now referring to FIG. 14, the dynamic connector 100 may further define hook-like concavities 118 and 118′ at each end portion of the elongate body 102. The concavities 118 and 118′ may be concave in shape and define a hook-like extension that is operable to grasp and secure the rods/prostheses 112, 112′ to the dynamic connector 100. In an exemplary operation, the length of the rods/prostheses 112, 112′ are adjusted to a desired length within the dynamic connector 100 depending on the length of the spinal segment to be treated and/or the size of the overall assembly the dynamic connector 100 is affixed to. The concavities 118 and 118′ operate to secure the rods/prostheses 112, 112′ to the elongate body 102 at the selected length, which may be further adjusted to secure the assembly once the rods or prostheses are engaged with the concavities 118 and 118′. The joints 114 and 114′ may also be tightened or locked to define a particular range of motion of the assembly.

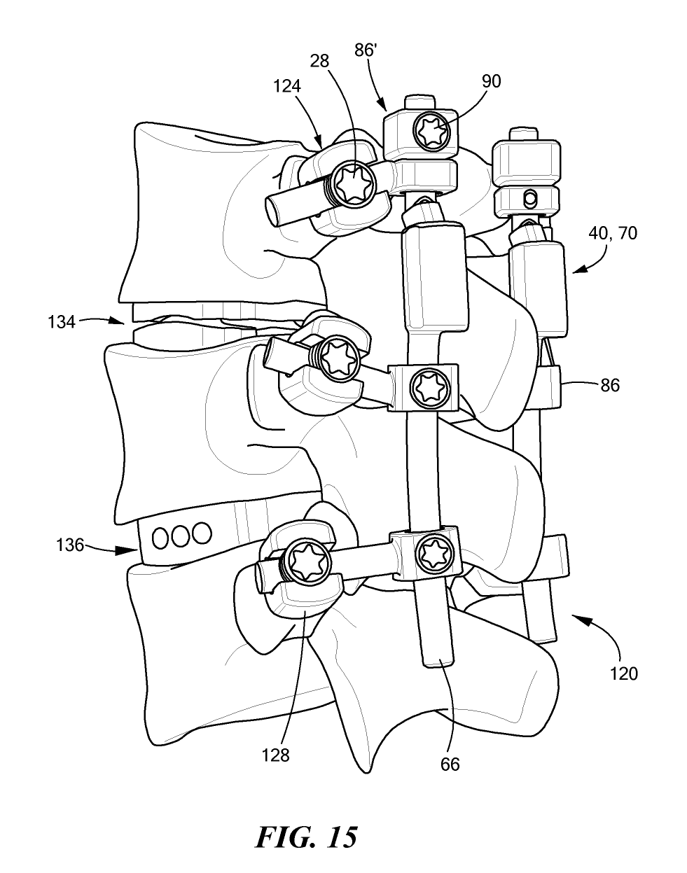

Now referring to FIG. 15, a dynamic hybrid spinal stabilization system 120 is shown. The hybrid stabilization system 120 may generally include one or more posterior stabilizing elements having a designated or selected range of motion, such as the dynamic stabilization devices 40, 70 and dynamic connectors 10, 90 and/or 100 described above. The hybrid stabilization system 120 can include tiers of dynamic stabilization devices 40 and/or 70 and dynamic connectors 90 and/or 100 that provide a desired degree of normal or physiological movement, while also fusing or affixing selected portions of the spinal segment.

The hybrid stabilization system 120 may further include a plurality of affixation elements 28, such as one or more pedicle screws, bone screws or the like, coupled to the pedicles. In an exemplary embodiment, two substantially parallel sets of three affixation elements 28 are anchored into consecutive pedicles, vertebrae or other portions of a spinal segment. For example, an exemplary spinal segment includes three vertebrae and at least two intervertebral spaces. One or more dynamic stabilization devices 40 are positioned on opposite sides (e.g., medial and lateral) of the spinal segment and are connected to at least one of the affixation elements 28. The dynamic stabilization devices 40 and/or 70 and dynamic connectors 90 and/or 100 may provide rotational, lateral, and translational motion and are spaced medial to the affixation elements 28.

Each affixation element 28 may receive the connector element 86 or 86′ extending medially from the affixation element 28 to the dynamic stabilization devices 40 and/or 70 and dynamic connectors 90 and/or 100. The connector elements 86 or 86′ may operate to rigidly or movably affix the dynamic stabilization devices 40 and/or 70 and dynamic connectors 90 and/or 100 to the affixation element 12. The affixation elements 28 may define a path for insertion of the connector elements 86 or 86′ such that the connector elements 86 or 86′ are slideably received within the path as discussed below. Once the connector elements 86 or 86′ are inserted within the path, a set screw or the like may be inserted within the affixation element 28 to immobilize the connector elements 86. The intersection between the connector elements 86 or 86′ and the dynamic stabilization devices 40 and/or 70 and dynamic connectors 90 and/or 100 may form an acute angle, or any angle depending on the desired affixation points of the respective components. For example, the connector elements 86 or 86′ may be affixed to the dynamic stabilization devices 40 and/or 70 and dynamic connectors 90 and/or 100 at a desired angle. The affixation element 28 may then immovably affix connector elements 86 or 86′ to the pedicle at the predetermined angle.

To further aid in the attachment of the connector elements 86 or 86′ and the affixation element 28, a head portion 124 of the affixation element 28 is operable to receive a portion of connector elements 86 or 86′. Once received within the head portion 124, a set screw or other similar fastener, may secure the connector elements 86 or 86′ to the affixation element 28. The head portion 124 may include two diametrically opposed curved sections, each of which substantially surround the connector elements 86 or 86′, and a flat section, such that the head portion 124 as a whole is substantially U-shaped. The head portion 124 may operate to facilitate or rotation and/or movement of the connector elements 86 or 86′ within affixation element 28. In an exemplary embodiment, three vertically spaced pairs of affixation elements 28 are affixed to the pedicles or vertebrae along a section of the spinal column, each affixation element 28 having the connector elements 86 or 86′ extending from it, and a head portion 124 partially surrounding it.

One or more intervertebral prostheses, including a paired articulating intervertebral disc prosthesis 134 may also be used in conjunction with the hybrid system 120 to provide a complete implantation system to remedy a degenerative or unhealthy spinal segment. Additionally, one or more fusion cages or other non-articulating prostheses 136 may be interposed between two vertebrae to further limit the range of motion while stabilizing the overall spinal segment.

In an exemplary use, the system 120 is affixed to a spinal segment having three vertebrae and two respective intervertebral spaces. The stabilization device 40 is positioned such that it spans a first intervertebral space defined by the first and second vertebrae, where the first vertebra is superior to the second vertebra. The first attachment element 66 of stabilization device 40 (or attachment element 84 for stabilization device 70) may be attached to an anchoring element 128 by either of the motion-providing connector element 86′ or the fixated connector element 86 to provide a selected movement as discussed above in the span of first intervertebral space. The second attachment element 66′, however, spans the length of the second and third vertebrae and may be attached to anchoring elements 128 in the second and third vertebrae the fixated connector element 86 to limit or altogether prevent movement between the second and third vertebrae. In this sense, the stabilization device 40 (or 70) provides a first segment (e.g., the first and second components movable with respect to one another) that allows articulation between first and second adjacent vertebra while also providing a second segment (e.g., the elongated second attachment element 66′) that prevents movement between second and third adjacent vertebra. The result of this configuration is that a selectable range of motion can be provided between the first and second vertebra while motion is restricted or prevented between the second and third vertebra.

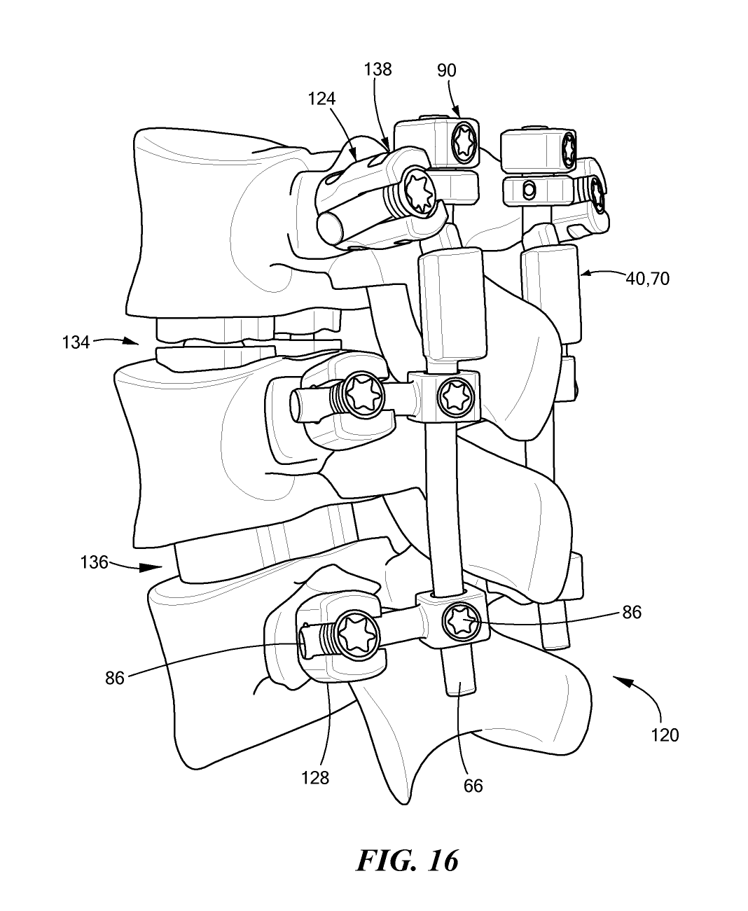

Now referring to FIG. 16, an alternative embodiment of the hybrid stabilization system 120 is shown. In particular, one or more of the affixation elements 28 may be replaced by one or more dynamic anchoring elements 138 that may allow some translational and rotational movement. For example, two of the six affixation elements 28 shown in FIG. 15, may be replaced with two dynamic anchoring elements 138. The dynamic anchoring elements 138 may allow the connector elements 86 or 86′ to move within the head portion 124 by defining a joint, for example, between the affixation element 28 and the connector elements 86 or 86′, which may increase the overall range of movement of the patient when the hybrid stabilization system 120 is installed. The remainder of the components describe with respect to FIG. 15 may be utilized in this embodiment.

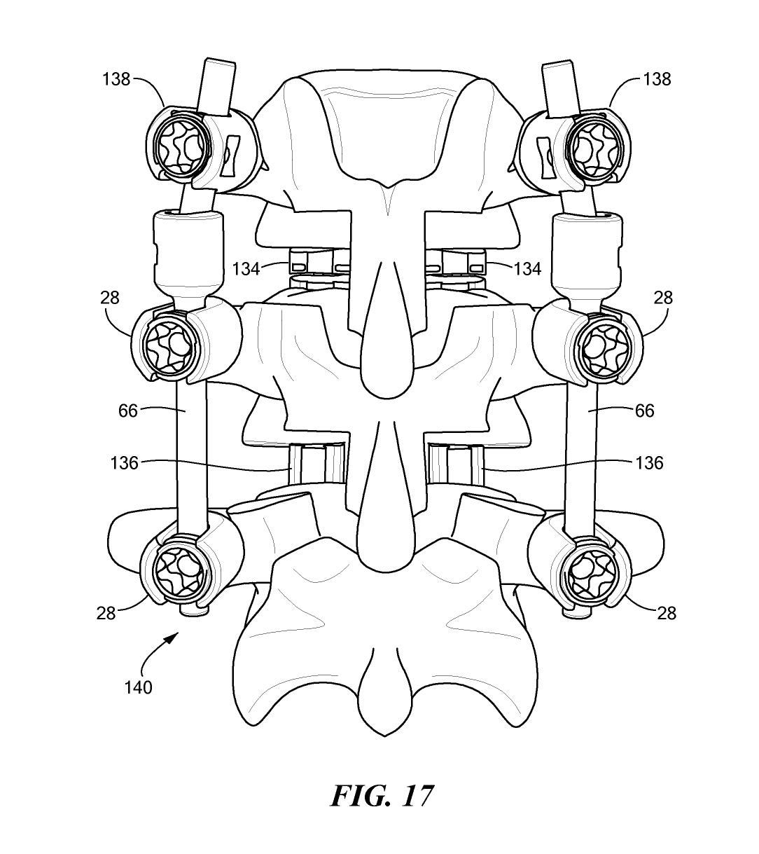

Now referring to FIG. 17, an alternative hybrid stabilization system 140 is shown. The connector elements 86 or 86′ are removed from the system 120 shown in FIGS. 15 and 16, and the attachment elements 66 or 66′ and the stabilization device 40 are affixed directly to the affixation elements in a more lateral position than shown in FIGS. 15 and 16. In this configuration, one or more of the attachment elements 66 or 66′ are movably affixed to the dynamic anchoring elements 138 and the stabilization device 40, and are immovably affixed to one or more affixation elements 28 and are each aligned substantially parallel to each other. The attachment elements 66 or 66′ may be angled or curved to conform to the shape of the pedicles and to provide for increased natural movement. The remainder of the components described with respect to FIG. 15 may be also be utilized in this configuration.

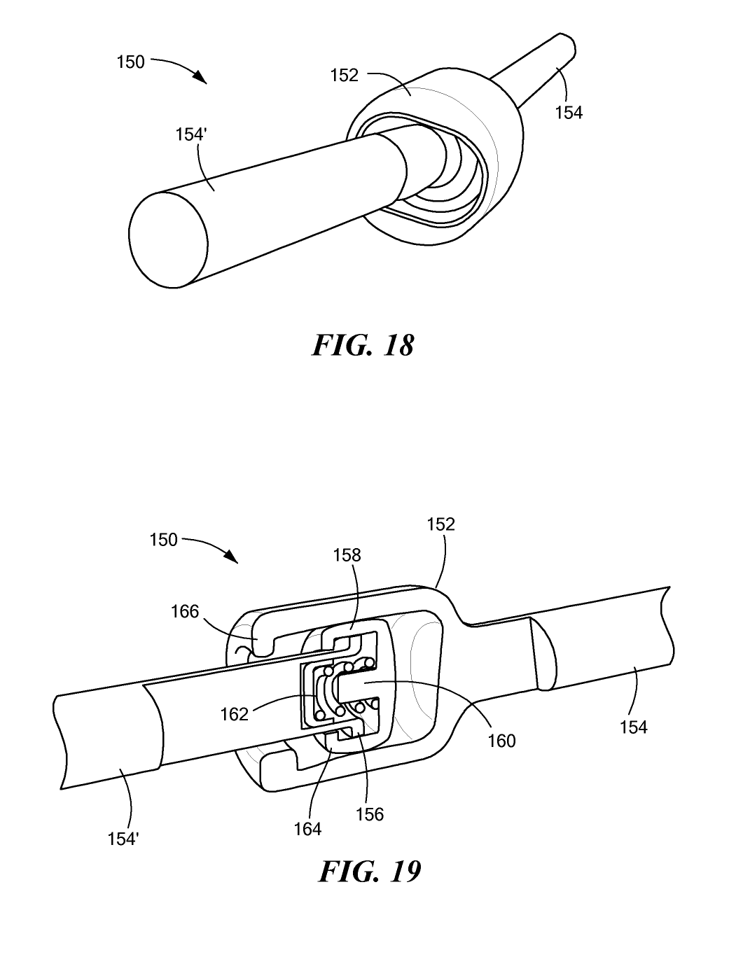

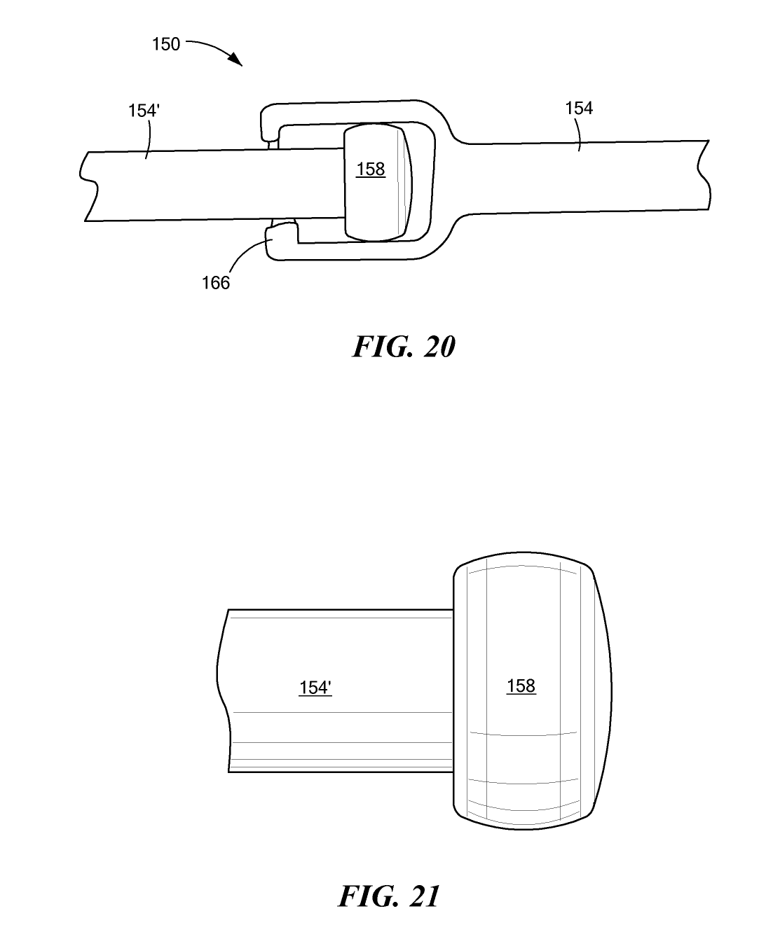

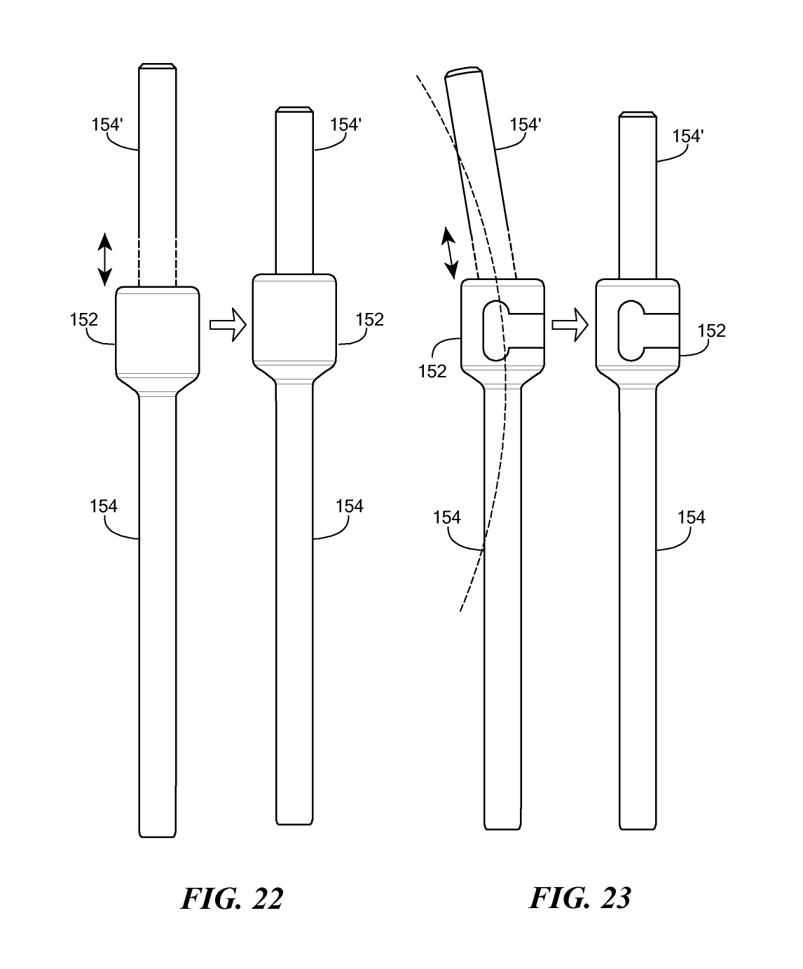

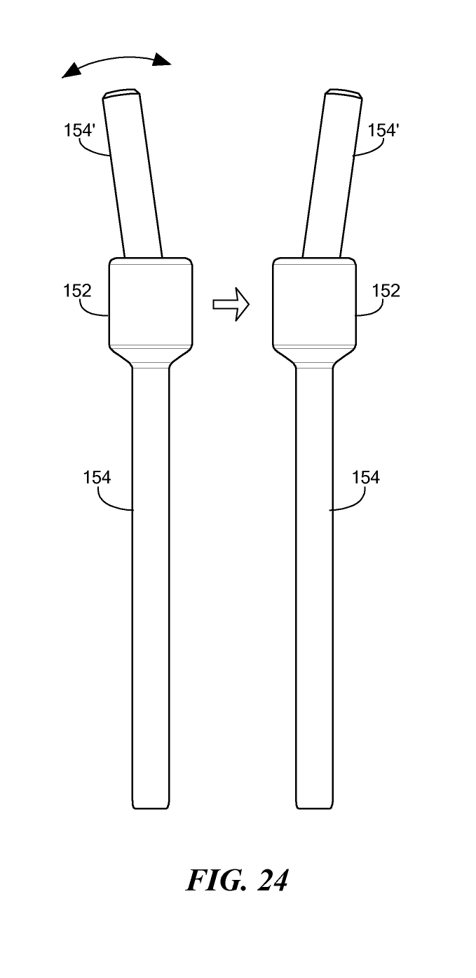

Now referring to FIGS. 18-24, another embodiment of a dynamic stabilization device 150 is shown that may be used as part of the hybrid stabilization systems 120 (FIGS. 15-16) and 140 (FIG. 17) discussed above. The dynamic stabilization device 150 may include a housing 152, which may substantially cylindrical in shape, and defines a hollow interior. A pair of attachment elements 154 and 154′, extend from the housing 152 in the inferior and superior directions respectively. For example, the housing 152 may define an opening at its proximal end for receiving attachment element 154′, which allows for some degree of rotational movement, and may be fused or otherwise coupled to attachment element 154 at its distal end.

The attachment element 154′ may define flared edges 156 at its proximal end, the proximal end of the attachment element 154′ being disposed within the housing 152. For example, as shown in FIG. 19, the flared edges define a substantially U-shaped portion at the distal end of the attachment element 154′. The proximal ends of either or both attachment elements 154 and 154′ may define a substantially convex surface, which may operate to resist angular motion of the device 150. The flared edges 156 are disposed within and contact a bearing 158 disposed within the housing 152. The bearing 158 may be substantially disc-shaped and may define a substantially convex outer surface that contacts the inner surface of the housing 152 and may further define an aperture for receiving the attachment element 154′. The bearing 158 further defines an inner protrusion 160, which may be substantially cylindrical in shape and extends towards the attachment element 154. Surrounding the inner protrusion 160 is a tensioning element 162, such as a spring or a biased coil, which is in contact with the proximal end of the attachment element 154′ and the inner surface of the bearing 158. The tensioning element 162 operates to resist movement in the inferior and superior directions when the device 150 is in operation. The bearing element 158 further defines a pair of indentations 164 extending inward at the aperture.

In an exemplary operation of the device 150, when a force is applied to the device 150 in the superior direction, the bearing 158 is pulled towards and contacts a pair of ridges 166 defined by the housing 152 at the opening. As the superior force continues to be applied to the attachment element 154′, the tensioning element 162 will apply an increasingly greater force that resists the superior movement of the attachment element 154′ while the bearing 158 remains secured to the housing 152. The maximum superior movement of the attachment element 154′ is achieved when the flared edges 156 contact the indentations 164. Similarly, when a force is applied to the housing 152 in the inferior direction, the attachment element 154′ and the bearing 158 will move in the inferior direction towards the inner surface of the housing 152 until the bearing 158 contacts the inner surface of the housing 152. As the inferior force continues to be applied after the bearing 158 contacts the inner surface of the housing 152, the attachment element 154′ will continue to move in the inferior direction and the tensioning element 162 will apply an increasingly greater force that resists the inferior movement of the attachment element 154′. The maximum inferior movement of the attachment element 154′ is achieved when the flared edges 156 contact the inner surface of the bearing 158.

It will be appreciated by persons skilled in the art that the present invention is not limited to what has been particularly shown and described herein above. In addition, unless mention was made above to the contrary, it should be noted that all of the accompanying drawings are not to scale. A variety of modifications and variations are possible in light of the above teachings without departing from the scope and spirit of the invention, which is limited only by the following claims.

What is claimed is:

1. A spinal stabilization system, comprising:
a posterior stabilization device including a first segment engageable with a first and second vertebra, the first segment providing a range of motion allowing at least one of the first and second vertebra to articulate with respect to the other, and a second segment engageable with the second vertebra and a third vertebra, the second segment substantially preventing movement of the second and third vertebrae with respect to one another.

2. The spinal stabilization system according to claim 1, further comprising a plurality of affixation elements engageable with the posterior stabilization device to secure the posterior stabilization device to the first, second, and third vertebrae.

3. The spinal stabilization system according to claim 2, wherein at least one of the affixation elements defines a head movably coupled to a threaded shaft.

4. The spinal stabilization system according to claim 2, further comprising a connector element, the connector element defining a collar engageable with the posterior stabilization device and an elongate portion engageable with an affixation element.

5. The spinal stabilization system according to claim 4, wherein the connector element defines a joint providing movement between the collar and the stabilization device.

6. The spinal stabilization system according to claim 1, further comprising an articulating intervertebral prosthesis positionable between the first and second vertebrae.

7. The spinal stabilization system according to claim 6, further comprising a non-articulating intervertebral prosthesis positionable between the second and third vertebrae.

8. The spinal stabilization system according to claim 1, wherein the first segment of the posterior stabilization device defines an arcuate range of motion.

9. The spinal stabilization system according to claim 1, wherein the range of motion of the first segment of the posterior stabilization device is selectively adjustable.

10. The spinal stabilization system according to claim 1, wherein the first segment of the posterior stabilization device defines a joint having at least two degrees-of-freedom of movement.

11. The spinal stabilization system according to claim 1, wherein the first segment of the posterior stabilization device defines a joint having at least three degrees-of-freedom of movement.

12. The spinal stabilization system according to claim 1, wherein the first segment of the posterior stabilization device includes at least one resistive element providing resistance for at least a portion of the range of motion of the first segment.

13. The spinal stabilization system according to claim 1, wherein the second segment of the posterior stabilization device includes an elongate rod extending from the first segment.

14. The spinal stabilization system according to claim 1, wherein the first segment of the posterior stabilization device includes a first component movably coupled to a second component, and wherein the second segment of the stabilization device includes an elongated portion of the second component.

15. A spinal stabilization system, comprising:
a first posterior stabilization device including a first component engageable with a first vertebra and a second component engageable with a second vertebra, wherein the first and second components are movably coupled to one another to define a range of motion between the first and second vertebra, and wherein the second component is engageable with a third vertebra to substantially prevent movement between the second and third vertebrae;
a first affixation element anchoring the first component to the first vertebra;
a second affixation element anchoring the second component to the second vertebra; and
a third affixation element anchoring the second component to the third vertebra.

16. The spinal stabilization system according to claim 15, wherein at least one of the affixation elements defines a head movably coupled to a threaded shaft.

17. The spinal stabilization system according to claim 15, further comprising a connector element, the connector element defining a collar movably coupled to the first component and an elongate portion coupled to the first affixation element.

18. The spinal stabilization system according to claim 15, further comprising at least one of an articulating intervertebral prosthesis positionable between the first and second vertebrae and a non-articulating intervertebral prosthesis positionable between the second and third vertebrae.

19. The spinal stabilization system according to claim 15, wherein the first and second components are movably coupled to one another to define a joint having at least three degrees-of-freedom.

20. A spinal stabilization system, comprising:
a first posterior stabilization device including a first component engageable with a first vertebra and a second component engageable with a second vertebra, wherein the first and second components are movably coupled to one another to define a range of motion between the first and second vertebra, and wherein the second component is engageable with a third vertebra to substantially prevent movement between the second and third vertebrae;
a first affixation element anchoring the first component to the first vertebra;
a second affixation element anchoring the second component to the second vertebra;
a third affixation element anchoring the second component to the third vertebra;
a second posterior stabilization device including a third component engageable with the first vertebra and a fourth component engageable with the second vertebra, wherein the third and fourth components are movably coupled to one another to define a range of motion between the first and second vertebra, and wherein the fourth component is engageable with the third vertebra to substantially prevent movement between the second and third vertebrae;
a fourth affixation element anchoring the third component to the first vertebra;
a fifth affixation element anchoring the fourth component to the second vertebra; and
a sixth affixation element anchoring the fourth component to the third vertebra.
